(19) United States
(12) Patent Application Publication
(10) Pub. No.: US 2009/0270755 A1
(43) Pub. Date: Oct. 29, 2009

(54) PEDOMETER FOR THE BRAIN

(75) Inventors: Mary P. Czerwinski, Woodinville, WA (US); Kenneth D. Ray, Seattle, WA (US); Steven Bathiche, Kirkland, WA (US); Hong Choing, Collegeville, PA (US)

Correspondence Address: LEE & HAYES, PLLC, 601 W. RIVERSIDE AVENUE, SUITE 1400, SPOKANE, WA, 99201, US

(73) Assignee: MICROSOFT CORPORATION, Redmond, WA (US)

(21) Appl. No.: 12/111,408

(22) Filed: Apr. 29, 2008

(51) Int. Cl. A61B 5/04; H04L 9/32

(52) U.S. Cl. 600/545; 713/182

(57) ABSTRACT

The claimed subject mater provides systems and/or techniques that provide biometric feedback monitoring of brain activity. The system includes mechanisms that obtain indication of brain activity associated with an individual which can be utilized to ensure that the individual is maximizing his or her brain activity. Where it is determined that the individual is not optimally utilizing his or her brain, feedback can be directed to the individual in order to stimulate brain activity in a specified response center of the brain.

### BACKGROUND

As people age they tend to loose their mental acuity; their ability to recall details, once taken for granted, gradually diminishes. Nevertheless, recent research indicates that if people keep mentally active, constantly stimulating the entire brain rather than selective portions, the overall mental well being of the individual improves thereby staving off one of the deleterious effects of aging.

The subject matter as claimed is directed toward resolving or at the very least mitigating, one or all the problems elucidated above.

### SUMMARY

The following presents a simplified summary in order to provide a basic understanding of some aspects of the disclosed subject matter. This summary is not an extensive overview, and it is not intended to identify key/critical elements or to delineate the scope thereof. Its sole purpose is to present some concepts in a simplified form as a prelude to the more detailed description that is presented later.

In accordance with an aspect, the claimed subject matter provides machine implemented systems and/or methods that provide biometric feedback monitoring of brain activity. Generally, as a person ages, their brain capacity becomes more diminished. Thus, the matter disclosed and claimed herein can allow an individual to ensure that their brain is participating in sufficient exercises (e.g., tests, crosswords, word searches, brain games, etc.). Moreover, the claimed subject matter can monitor mental-health and thoughts (e.g. positive thoughts and causes, negative thoughts and causes, etc.). By tracking such brain activity and sensations, the claimed subject matter can collect feedback on how different things affect the person or individual.

In accordance with yet a further aspect of the claimed subject matter, since most people react to stimulation in an unconscious manner (e.g., people usually do not think about what they are doing and whether they enjoy or dislike an activity), the claimed subject matter can record the actions/non-actions an individual is taking (e.g., regardless if reactionary, consciously, or unconsciously, etc.). Moreover, in a further aspect, the claimed subject matter can be employed as a recording device that can continuously record what the individual is doing as well any ambient variables associated with the individual (e.g., factors surrounding the individual's daily activities).

To the accomplishment of the foregoing and related ends, certain illustrative aspects of the disclosed and claimed subject matter are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a machine-implemented system that provides biometric feedback monitoring of brain activity in accordance with an aspect of the claimed subject matter.

FIG. 2 provides a more detailed depiction of an illustrative pedometer component that provides biometric feedback monitoring of brain activity in accordance with an aspect of the claimed subject matter.

FIG. 3 provides a more detailed depiction of an illustrative analysis component that provides biometric feedback monitoring of brain activity in accordance with an aspect of the claimed subject matter.

FIG. 4 illustrates a system implemented on a machine that provides biometric feedback monitoring of brain activity in accordance with an aspect of the claimed subject matter.

FIG. 5 provides a further depiction of a machine implemented system that provides biometric feedback monitoring of brain activity in accordance with an aspect of the claimed subject matter.

FIG. 6 illustrates yet another aspect of the machine implemented system that provides biometric feedback monitoring of brain activity in accordance with an aspect of the claimed subject matter.

FIG. 7 depicts a further illustrative aspect of the machine implemented system that provides biometric feedback monitoring of brain activity in accordance with an aspect of the claimed subject matter.

FIG. 8 illustrates another illustrative aspect of a system implemented on a machine that provides biometric feedback monitoring of brain activity in accordance with an aspect of the claimed subject matter.

FIG. 9 depicts yet another illustrative aspect of a system that provides biometric feedback monitoring of brain activity in accordance with an aspect of the claimed subject matter.

FIG. 10 illustrates a flow diagram of a machine implemented methodology that provides biometric feedback monitoring of brain activity in accordance with an aspect of the claimed subject matter.

FIG. 11 illustrates a block diagram of a computer operable to execute the disclosed system in accordance with an aspect of the claimed subject matter.

FIG. 12 illustrates a schematic block diagram of an illustrative computing environment for processing the disclosed architecture in accordance with another aspect.

### DETAILED DESCRIPTION

The subject matter as claimed is now described with reference to the drawings, wherein like reference numerals are used to refer to like elements throughout. In the following description, for purposes of explanation, numerous specific details are set forth in order to provide a thorough understanding thereof. It may be evident, however, that the claimed subject matter can be practiced without these specific details. In other instances, well-known structures and devices are shown in block diagram form in order to facilitate a description thereof.

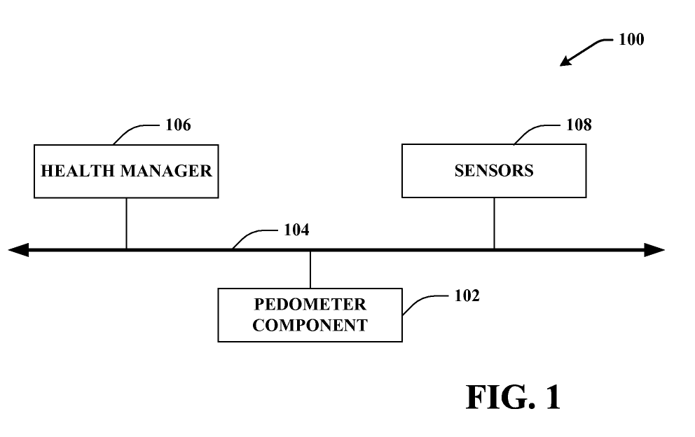

FIG. 1 illustrates a machine implemented system 100 that provides biometric feedback monitoring of brain activity in accordance with an aspect of the claimed subject matter. System 100 can include pedometer component 102 that accesses or acquires data and/or information from a multitude of sources including health manager 106 and/or sensors 108 via network topology and/or cloud 104. Pedometer component 102 can utilize received or obtained information to control or regulate aspects of a users' behavior (e.g., inducing the individual to partake in mentally challenging activities, such as numeric games, crossword puzzles, memorizing lists, as well as extorting the individual to partake in physical activities that involve or include mentally challenging aspects, such are orienteering). Additionally and/or alternatively, pedometer component 102 through use of multiple sensors (e.g., sensors 108) can ascertain that the whole brain partakes in some form of activity thereby ensuring the well being of the brain in its entirety.

Network topology and/or cloud 104 can include any viable communication and/or broadcast technology, for example, wired and/or wireless modalities and/or technologies can be utilized to effectuate the claimed subject matter. Moreover, network topology and/or cloud 104 can include utilization of Personal Area Networks (PANs), Local Area Networks (LANs), Campus Area Networks (CANs), Metropolitan Area Networks (MANs), extranets, intranets, the Internet, Wide Area Networks (WANs)—both centralized and/or distributed—and/or any combination, permutation, and/or aggregation thereof. Additionally, network topology and/or cloud 104 can include or encompass communications or interchange utilizing Near-Field Communications (NFC) and/or communications utilizing electrical conductance of the human skin, for example.

Health manager 106 can be an online repository and/or directed search facility that persists or stores an individual's health data ranging from test results to physician's reports to daily measurements of weight or blood pressure. Individuals can then have access to their records at any time, anywhere, via network topology and/or cloud 104. Affiliated medical practitioners, medical offices, and/or hospitals can, for instance, easily forward test results in digital form to health manager 106, and individuals (e.g. patients) can in turn authorize selected medical practitioners, medical offices, hospitals, components owned or controlled by the individual (e.g., pedometer component 102), and the like, to access various carefully circumscribed aspects of their personal data. Moreover, health manager 106 can provide centralized storage and access to personal health records so that users can easily access their personal records from anywhere and at anytime regardless of the access platform (e.g., cell phone, smart phone, gaming console, and the like). Further, pedometer component 102 can access health manger 106 based at least in part on a unique user identity wherein the user identity can be associated with a network identity or online gaming persona, or the user identity can be acquired through a token persisted on portable flash devices (e.g., card or Universal Serial Bus (USB) flash devices).

Additionally and/or alternatively, health manager 106 can also provide directed and/or targeted vertical search capabilities that can provide more relevant results than generalist search engines. For instance, a search actuated on health manager 106 can allow individuals to specifically tailor their search queries based on their persisted health records, past queries, and the like, and can receive in return results that are most relevant to each individual's situation. In addition, an offline portable record store can act as health manager 106, wherein pedometer component 102 can call into functions exposed on active portable record stores (e.g., when the portable record store is online). For instance, a cell phone can have persisted personal records and can respond to service calls (e.g., via Bluetooth) to provide or extract records from associated flash memory.

Sensors 108 can be any mechanism or device that can be utilized to measure or observe activity or inactivity, or dietary habits associated with a particular individual. Sensors 108 can include, without limitation, microphones, cameras, pedometers, accelerometers, brain probes or electrodes that measure the electrical activity of the brain and/or brain scan monitors, heart rate monitors, thermometers, blood sugar monitors, devices associated with exercise machines such as elliptical machines, treadmills, exercise bicycles, step machines, devices incorporated into running tracks, swimming pools, basketball courts, and the like, devices utilized in home and/or office environments to monitor ambient variables (e.g., thermostats, motion detectors, and the like), or any other device or component that can be utilized to monitor activity or inactivity associated with users of system 100. Further, sensors 108 can be included with, or incorporated in, textiles, fabrics, clothing, jewelry, or any item that can be worn.

Additionally, sensors 108 can be Radio Frequency Identification (RFID) devices, or can incorporate or subsume Radio Frequency Identification (RFID) devices. For example, sensors 108 can be a wrist band that has embedded therein a Radio Frequency Identification (RFID) tag or chip that can continuously obtain information (e.g., electrical impulses from the brain, blood pressure, body temperature, perspiration, pupil dilation, heart rate, location information from global positioning systems, . . . ) from the wearer of the wrist band. Additionally, sensors 108 can be implemented entirely in hardware and/or as a combination of hardware and/or software in execution. Further, sensors 108 can be any type of mechanism, machine, device, facility, and/or instrument that includes a processor and is capable of effective and/or operative communication with network topology and/or cloud 104. Illustrative mechanisms, machines, devices, facilities, and/or instruments of conversion that can comprise sensors 108 can include Tablet PCs, server class computing machines, laptop computers, notebook computers, desktop computers, cell phones, smart phones, consumer appliances and/or instrumentation, industrial devices and/or components, hand-held devices, personal digital assistants, multimedia Internet enabled phones, Global Positioning Systems (GPS), USB flash devices, multimedia players, and the like.

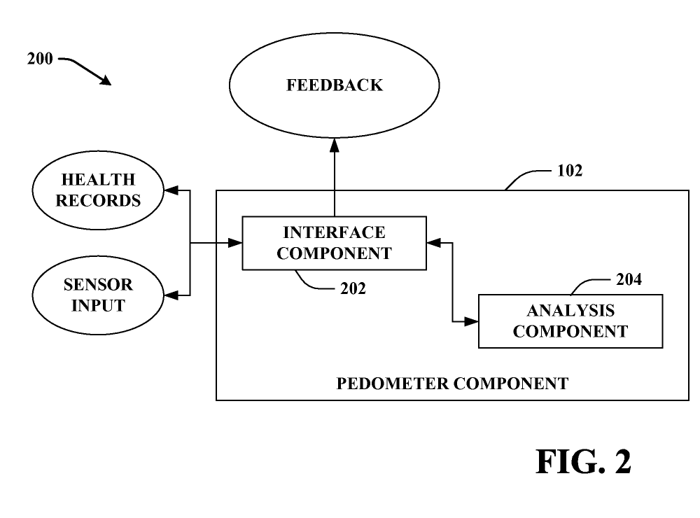

FIG. 2 provides further illustration 200 of an illustrative pedometer component 102 in accordance with an aspect of the claimed subject matter. As illustrated pedometer component 102 can actively and/or passively acquire or access input, such as, for example, input from sensors 108 and/or health related records from health manager 106 via interface component 202. Interface component 202 (hereinafter referred to as “interface 202”) can receive and/or disseminate, communicate, and/or partake in data interchange with a plurality of disparate sources and/or components. For instance, interface 202 can receive and/or transmit data from, or to, a multitude of sources, such as, for example, data associated with health records obtained from health manager 106, and brain activity levels obtained from and observed by sensors 108. Additionally and/or alternatively, interface 202 can obtain and/or receive data associated with usernames and/or passwords, sets of encryption and/or decryption keys, client applications, services, users, clients, devices, and/or entities involved with a particular transaction, portions of transactions, and thereafter can convey the received or otherwise acquired information to analysis component 204, for subsequent and further utilization, processing, and/or analysis.

To facilitate its objectives, interface 202 can provide various adapters, connectors, channels, communication pathways, etc. to integrate the various components included in system 200, and more particularly, pedometer component 102, into virtually any operating system and/or database system and/or with one another. Additionally and/or alternatively, interface 202 can provide various adapters, connectors, channels, communication modalities, and the like, that can provide for interaction with the various components that can comprise system 200, and/or any other component (external and/or internal), data, and the like, associated with system 200.

Analysis component 204 can utilize inputs such as health records from health manager 106 and information gleaned from sensors 108 to ascertain brain activity, monitor the health of the brain (e.g., ensure that all parts of the brain are being utilized on a regular basis), and determine what the individual is thinking about at any instance in time based on where the individual is located (e.g., typically there is a correlation between an individuals surroundings and their thoughts; the more pleasant the surroundings the more settled the individual's thoughts).

Further, analysis component 204 can keep track of brain activity and/or sensations experienced by the individual when exposed to different stimuli. Additionally, positive reactions and/or graded aversions to selected stimuli can be noted and/or recorded by analysis component 204. Keeping track of, or recording, brain activity and/or sensations experienced in response to stimuli can be beneficially employed to provide feedback to the individual when tasks are assigned by the claimed subject matter in order to target selected response centers of the brain.

Moreover, analysis component 204 can continuously record sources of stimuli (e.g., from ambient monitors or sensors, such as sensors 108, associated with, or dispersed around, the individual). Analysis component 204 through elicited or received inputs (e.g., health records, sensor input, etc.) can also investigate how much time the individual dwells on a particular sensation or thought (e.g., incessantly dwelling on particular thoughts and/or sensations can be an indication of depression or some other brain abnormality).

Formulated output from analysis component 204 in the form of feedback can be utilized to enhance electronic gaming activities whereby the feedback can inform the electronic game so that game characteristics (including attributes associated with a particular game character) can be dynamically shifted or changed based at least in part on the level of activity or inactivity detected by the claimed subject matter, and in particular pedometer component 102.

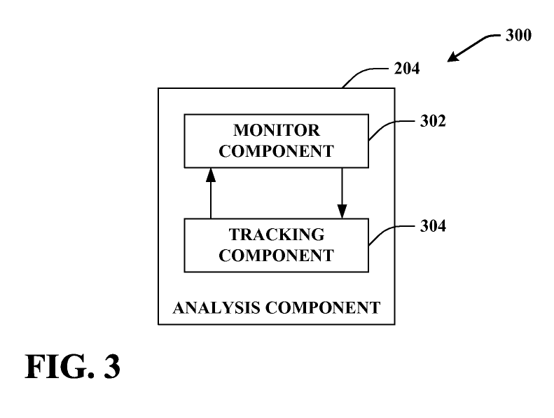

FIG. 3 provides depiction 300 of an illustrative analysis component 204 in accordance with an aspect of the claimed subject matter. Analysis component 204 as illustrated can include monitor component 302 that monitors brain activity via input from sensors 108 and utilization of health records (e.g., previous baseline electroencephalography (EEG) measurements, magnetoencephalography (MEG) images, CAT scans of the brain, etc.) obtained from health manager 106. Such input can be utilized to ensure that an individual's brain in its entirety is being utilized on a regular basis. Moreover, monitor component 302 can be utilized to monitor the health of the brain by ensuring that the individual is not dwelling on particularly negative or morbid thoughts (e.g., an indication of depression) in which case the subject matter as claimed can be used to shift the individual's focus away from the detected thoughts to more pleasant endeavors and/or past times (e.g., by suggesting the individual play a video game, go for a walk, watch a movie, . . . ).

Additionally, analysis component 204 can include tracking component 304 that keeps track of brain activity and/or sensations experienced by the individual during the course of the day. Reactions, both positive and negative, can be noted and/or recorded by tracking component 304 and such reactions can be utilized to aid the individual to target selected response centers of the brain. Moreover, tracking component 304 can also record sources of stimulus which can once again be utilized to aid the individual to activate specific response centers of the brain. For instance, it might be noted that certain activities (e.g., arithmetical computations) atypically stimulate one portion of a particular individual's brain. Where this is the case tracking component 304 can ensure that pedometer component 102 is made aware of this anomaly and as such when pedometer component 102 needs to activate those portions of that individual's brain it can find or devise challenging arithmetical computations to satisfy this need.

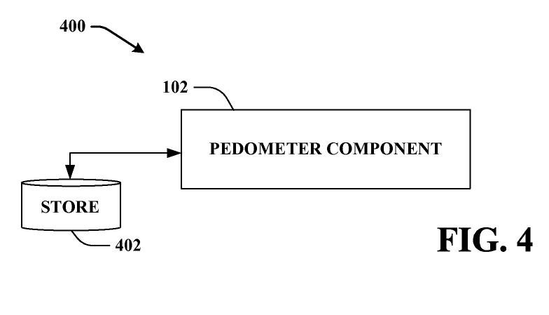

FIG. 4 depicts an aspect of a system 400 that provides biometric feedback monitoring of brain activity in accordance with an aspect of the claimed subject matter. System 400 can include store 402 that can include any suitable data necessary for pedometer component 102 to facilitate it aims. For instance, store 402 can include information regarding user data, data related to a portion of a transaction, credit information, historic data related to a previous transaction, a portion of data associated with purchasing a good and/or service, a portion of data associated with selling a good and/or service, geographical location, online activity, previous online transactions, activity across disparate networks, activity across a network, credit card verification, membership, duration of membership, communication associated with a network, buddy lists, contacts, questions answered, questions posted, response time for questions, blog data, blog entries, endorsements, items bought, items sold, products on the network, information gleaned from a disparate website, information obtained from the disparate network, ratings from a website, a credit score, geographical location, a donation to charity, or any other information related to software, applications, web conferencing, and/or any suitable data related to transactions, etc.

It is to be appreciated that store 402 can be, for example, volatile memory or non-volatile memory, or can include both volatile and non-volatile memory. By way of illustration, and not limitation, non-volatile memory can include read-only memory (ROM), programmable read only memory (PROM), electrically programmable read only memory (EPROM), electrically erasable programmable read only memory (EEPROM), or flash memory. Volatile memory can include random access memory (RAM), which can act as external cache memory. By way of illustration rather than limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink® DRAM (SLDRAM), Rambus® direct RAM (RDRAM), direct Rambus® dynamic RAM (DRDRAM) and Rambus® dynamic RAM (RDRAM). Store 402 of the subject systems and methods is intended to comprise, without being limited to, these and any other suitable types of memory. In addition, it is to be appreciated that store 402 can be a server, a database, a hard drive, and the like.

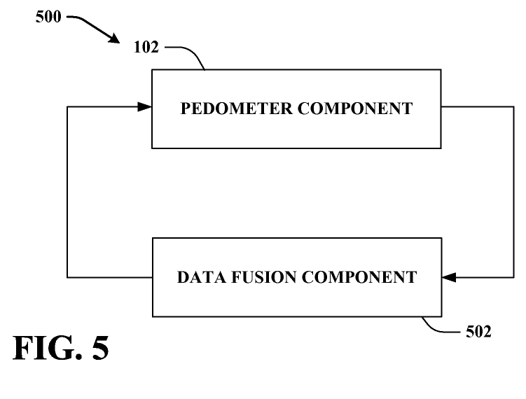

FIG. 5 provides yet a further depiction of a system 500 that provides biometric feedback monitoring of brain activity in accordance with an aspect of the claimed subject matter. As depicted, system 500 can include a data fusion component 502 that can be utilized to take advantage of information fission which may be inherent to a process (e.g., receiving and/or deciphering inputs) relating to analyzing inputs through several different sensing modalities. In particular, one or more available inputs may provide a unique window into a physical environment (e.g., an entity inputting instructions) through several different sensing or input modalities. Because complete details of the phenomena to be observed or analyzed may not be contained within a single sensing/input window, there can be information fragmentation which results from this fission process. These information fragments associated with the various sensing devices may include both independent and dependent components.

The independent components may be used to further fill out (or span) an information space; and the dependent components may be employed in combination to improve quality of common information recognizing that all sensor/input data may be subject to error, and/or noise. In this context, data fusion techniques employed by data fusion component 502 may include algorithmic processing of sensor/input data to compensate for inherent fragmentation of information because particular phenomena may not be observed directly using a single sensing/input modality. Thus, data fusion provides a suitable framework to facilitate condensing, combining, evaluating, and/or interpreting available sensed or received information in the context of a particular application.

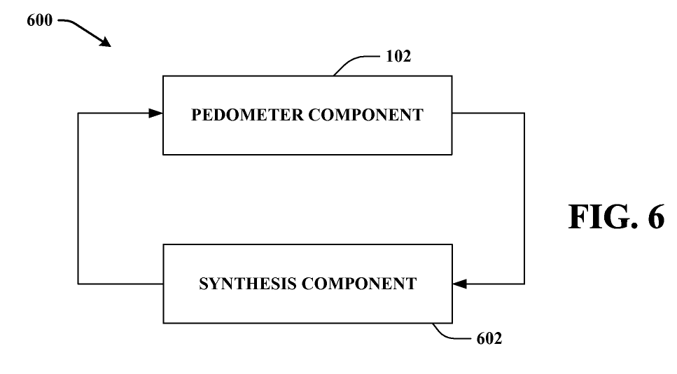

FIG. 6 provides a further depiction of a system 600 that provides biometric feedback monitoring of brain activity in accordance with an aspect of the claimed subject matter. As illustrated pedometer component 102 can, for example, employ synthesis component 602 to combine, or filter information received from a variety of inputs (e.g., text, speech, gaze, environment, audio, images, gestures, noise, temperature, touch, smell, handwriting, pen strokes, analog signals, digital signals, vibration, motion, altitude, location, GPS, wireless, etc.), in raw or parsed (e.g. processed) form. Synthesis component 602 through combining and filtering can provide a set of information that can be more informative, or accurate (e.g., with respect to an entity's communicative or informational goals) and information from just one or two modalities, for example. As discussed in connection with FIG. 5, the data fusion component 502 can be employed to learn correlations between different data types, and the synthesis component 602 can employ such correlations in connection with combining, or filtering the input data.

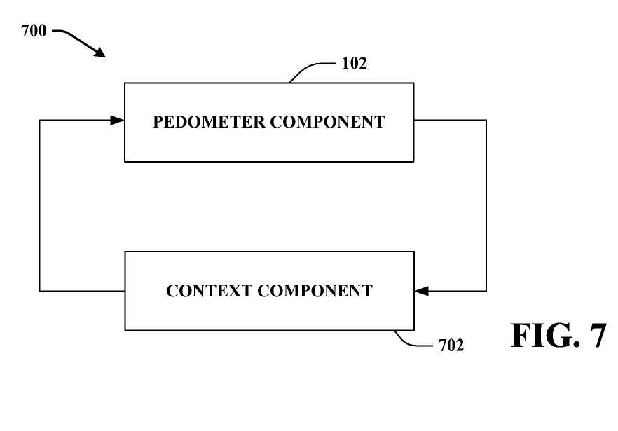

FIG. 7 provides a further illustration of a system 700 that provides biometric feedback monitoring of brain activity in accordance with an aspect of the claimed subject matter. As illustrated pedometer component 102 can, for example, employ context component 702 to determine context associated with a particular action or set of input data. As can be appreciated, context can play an important role with respect understanding meaning associated with particular sets of input, or intent of an individual or entity. For example, many words or sets of words can have double meanings (e.g., double entendre), and without proper context of use or intent of the words the corresponding meaning can be unclear thus leading to increased probability of error in connection with interpretation or translation thereof. The context component 702 can provide current or historical data in connection with inputs to increase proper interpretation of inputs. For example, time of day may be helpful to understanding an input—in the morning, the word “drink” would likely have a high a probability of being associated with coffee, tea, or juice as compared to being associated with a soft drink or alcoholic beverage during late hours. Context can also assist in interpreting uttered words that sound the same (e.g., steak and, and stake). Knowledge that it is near dinnertime of the user as compared to the user camping would greatly help in recognizing the following spoken words “I need a steak/stake”. Thus, if the context component 702 had knowledge that the user was not camping, and that it was near dinnertime, the utterance would be interpreted as “steak”. On the other hand, if the context component 702 knew (e.g., via GPS system input) that the user recently arrived at a camping ground within a national park; it might more heavily weight the utterance as “stake”.

In view of the foregoing, it is readily apparent that utilization of the context component 702 to consider and analyze extrinsic information can substantially facilitate determining meaning of sets of inputs.

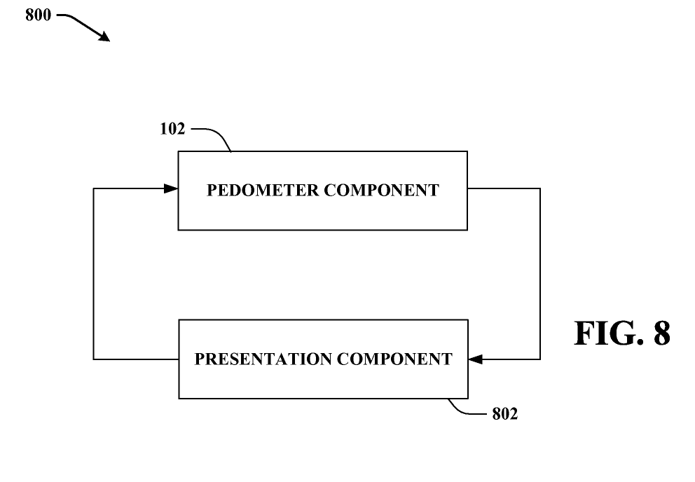

FIG. 8 provides further illustration of a system 800 that provides biometric feedback monitoring of brain activity in accordance with an aspect of the claimed subject matter. As illustrated, system 800 can include presentation component 802 that can provide various types of user interface to facilitate interaction between a user and any component coupled to pedometer component 102. Presentation component 802 can provide one or more graphical user interface, command line interface, and the like. For example, a graphical user interface can be rendered that provides the user with a region or means to load, import, read, etc., data, and can include a region to present the results of such. These regions can comprise known text and/or graphic regions comprising dialog boxes, static controls, drop-down menus, list boxes, pop-up menus, edit controls, combo boxes, radio buttons, check boxes, push buttons, and graphic boxes. In addition, utilities to facilitate the presentation such as vertical and/or horizontal scrollbars for navigation and toolbar buttons to determine whether a region will be viewable can be employed. For example, the user can interact with one or more of the components coupled and/or incorporated into pedometer component 102.

Users can also interact with regions to select and provide information via various devices such as a mouse, roller ball, keypad, keyboard, and/or voice activation, for example. Typically, mechanisms such as a push button or the enter key on the keyboard can be employed subsequent to entering the information in order to initiate, for example, a query. However, it is to be appreciated that the claimed subject matter is not so limited. For example, merely highlighting a checkbox can initiate information conveyance. In another example, a command line interface can be employed. For example, the command line interface can prompt (e.g., via text message on a display and/or an audio tone) the user for information via a text message. The user can then provide suitable information, such as alphanumeric input corresponding to an option provided in the interface prompt or an answer (e.g., verbal utterance) to a question posed in the prompt. It is to be appreciated that the command line interface can be employed in connection with a graphical user interface and/or application programming interface (API). In addition, the command line interface can be employed in connection with hardware (e.g., video cards) and/or displays (e.g., black-and-white, and EGA) with limited graphic support, and/or low bandwidth communication channels.

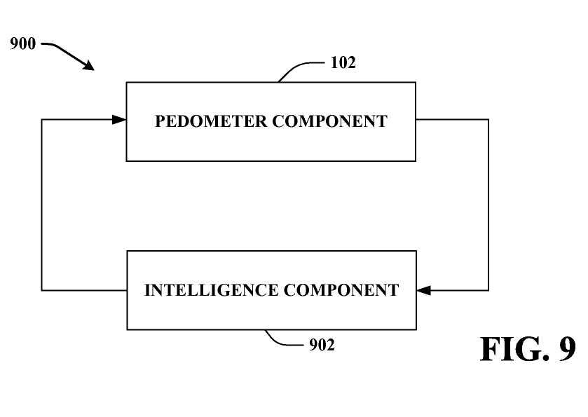

FIG. 9 depicts a system 900 that employs artificial intelligence to provide biometric feedback monitoring of brain activity in accordance with an aspect of the claimed subject matter. Accordingly, as illustrated, system 900 can include an intelligence component 902 that can employ a probabilistic based or statistical based approach, for example, in connection with making determinations or inferences. Inferences can be based in part upon explicit training of classifiers (not shown) before employing system 100, or implicit training based at least in part upon system feedback and/or users previous actions, commands, instructions, and the like during use of the system. Intelligence component 902 can employ any suitable scheme (e.g., neural networks, expert systems, Bayesian belief networks, support vector machines (SVMs), Hidden Markov Models (HMMs), fuzzy logic, data fusion, etc.) in accordance with implementing various automated aspects described herein. Intelligence component 902 can factor historical data, extrinsic data, context, data content, state of the user, and can compute cost of making an incorrect determination or inference versus benefit of making a correct determination or inference. Accordingly, a utility-based analysis can be employed with providing such information to other components or taking automated action. Ranking and confidence measures can also be calculated and employed in connection with such analysis.

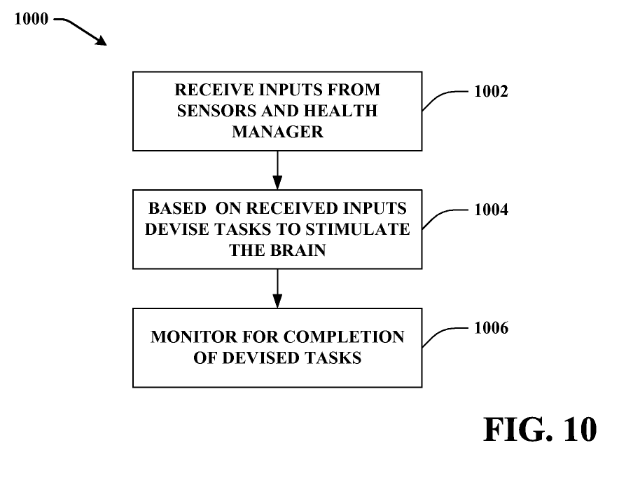

In view of the illustrative systems shown and described supra, methodologies that may be implemented in accordance with the disclosed subject matter will be better appreciated with reference to the flow chart of FIG. 10. While for purposes of simplicity of explanation, the methodologies are shown and described as a series of blocks, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methodologies described hereinafter. Additionally, it should be further appreciated that the methodologies disclosed hereinafter and throughout this specification are capable of being stored on an article of manufacture to facilitate transporting and transferring such methodologies to computers.

The claimed subject matter can be described in the general context of computer-executable instructions, such as program modules, executed by one or more components. Generally, program modules can include routines, programs, objects, data structures, etc. that perform particular tasks or implement particular abstract data types. Typically the functionality of the program modules may be combined and/or distributed as desired in various aspects.

FIG. 10 provides illustration of a machine implemented method 1000 that provides biometric feedback monitoring of brain activity in accordance with an aspect of the claimed subject matter. Method 1000 can commence at 1002 where inputs can be received from multiple sources such as brain activity acquired from sensors 108 and health records obtained from health manager 106. At 1004 the received input can be utilized to target selected response centers of the brain by devising tasks that specifically target the specified areas. For example, it may be noted that the frontal lobe of an individual has not been exercised for a period of time; accordingly, tasks can be identified and/or devised, based at least in part on health records, current and/or past sensor readings, and the like to activate the frontal lobe of the individual. At 1006 the individual's brain can be monitored to ascertain whether or not the individual has undertaken the prescribed and/or devised task assigned at 1004 (e.g., readings from sensors associated with the identified area of brain should indicate activity).

The claimed subject matter can be implemented via object oriented programming techniques. For example, each component of the system can be an object in a software routine or a component within an object. Object oriented programming shifts the emphasis of software development away from function decomposition and towards the recognition of units of software called “objects” which encapsulate both data and functions. Object Oriented Programming (OOP) objects are software entities comprising data structures and operations on data. Together, these elements enable objects to model virtually any real-world entity in terms of its characteristics, represented by its data elements, and its behavior represented by its data manipulation functions. In this way, objects can model concrete things like people and computers, and they can model abstract concepts like numbers or geometrical concepts.

As used in this application, the terms “component” and “system” are intended to refer to a computer-related entity, either hardware, a combination of hardware and software, or software in execution. For example, a component can be, but is not limited to being, a process running on a processor, a processor, a hard disk drive, multiple storage drives (of optical and/or magnetic storage medium), an object, an executable, a thread of execution, a program, and/or a computer. By way of illustration, both an application running on a server and the server can be a component. One or more components can reside within a process and/or thread of execution, and a component can be localized on one computer and/or distributed between two or more computers.

Artificial intelligence based systems (e.g., explicitly and/or implicitly trained classifiers) can be employed in connection with performing inference and/or probabilistic determinations and/or statistical-based determinations as in accordance with one or more aspects of the claimed subject matter as described hereinafter. As used herein, the term “inference,” “infer” or variations in form thereof refers generally to the process of reasoning about or inferring states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources. Various classification schemes and/or systems (e.g., support vector machines, neural networks, expert systems, Bayesian belief networks, fuzzy logic, data fusion engines . . . ) can be employed in connection with performing automatic and/or inferred action in connection with the claimed subject matter.

Furthermore, all or portions of the claimed subject matter may be implemented as a system, method, apparatus, or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device or media. For example, computer readable media can include but are not limited to magnetic storage devices (e.g., hard disk, floppy disk, magnetic strips . . . ), optical disks (e.g., compact disk (CD), digital versatile disk (DVD) . . . ), smart cards, and flash memory devices (e.g., card, stick, key drive . . . ). Additionally it should be appreciated that a carrier wave can be employed to carry computer-readable electronic data such as those used in transmitting and receiving electronic mail or in accessing a network such as the Internet or a local area network (LAN). Of course, those skilled in the art will recognize many modifications may be made to this configuration without departing from the scope or spirit of the claimed subject matter.

Some portions of the detailed description have been presented in terms of algorithms and/or symbolic representations of operations on data bits within a computer memory. These algorithmic descriptions and/or representations are the means employed by those cognizant in the art to most effectively convey the substance of their work to others equally skilled. An algorithm is here, generally, conceived to be a self-consistent sequence of acts leading to a desired result. The acts are those requiring physical manipulations of physical quantities. Typically, though not necessarily, these quantities take the form of electrical and/or magnetic signals capable of being stored, transferred, combined, compared, and/or otherwise manipulated.

It has proven convenient at times, principally for reasons of common usage, to refer to these signals as bits, values, elements, symbols, characters, terms, numbers, or the like. It should be borne in mind, however, that all of these and similar terms are to be associated with the appropriate physical quantities and are merely convenient labels applied to these quantities. Unless specifically stated otherwise as apparent from the foregoing discussion, it is appreciated that throughout the disclosed subject matter, discussions utilizing terms such as processing, computing, calculating, determining, and/or displaying, and the like, refer to the action and processes of computer systems, and/or similar consumer and/or industrial electronic devices and/or machines, that manipulate and/or transform data represented as physical (electrical and/or electronic) quantities within the computer's and/or machine's registers and memories into other data similarly represented as physical quantities within the machine and/or computer system memories or registers or other such information storage, transmission and/or display devices.

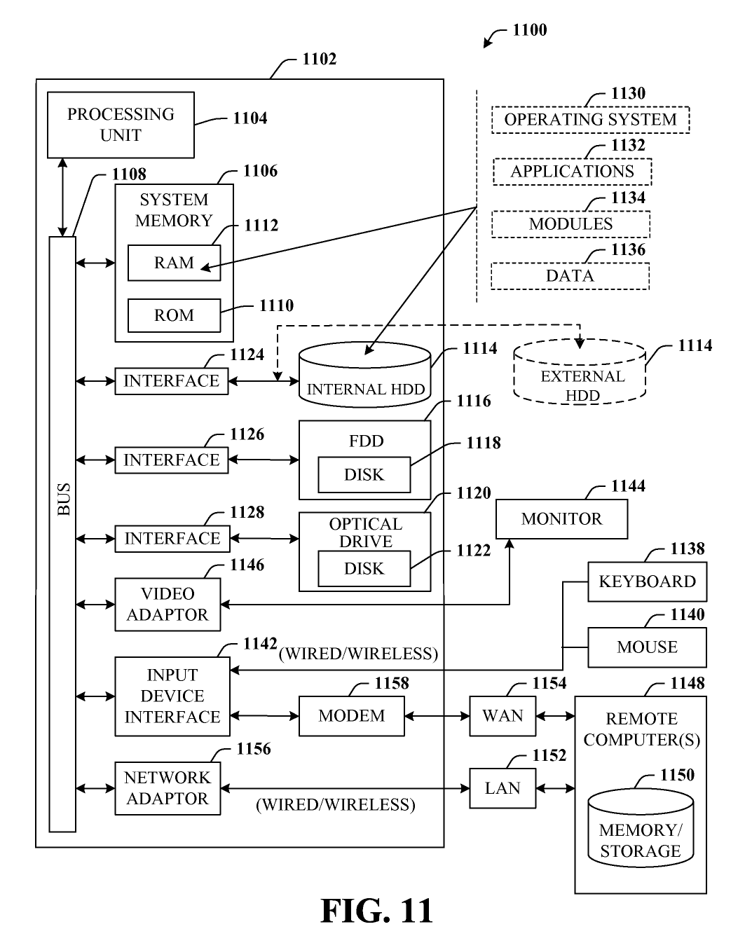

Referring now to FIG. 11, there is illustrated a block diagram of a computer operable to execute the disclosed system. In order to provide additional context for various aspects thereof, FIG. 11 and the following discussion are intended to provide a brief, general description of a suitable computing environment 1100 in which the various aspects of the claimed subject matter can be implemented. While the description above is in the general context of computer-executable instructions that may run on one or more computers, those skilled in the art will recognize that the subject matter as claimed also can be implemented in combination with other program modules and/or as a combination of hardware and software.

Generally, program modules include routines, programs, components, data structures, etc., that perform particular tasks or implement particular abstract data types. Moreover, those skilled in the art will appreciate that the inventive methods can be practiced with other computer system configurations, including single-processor or multiprocessor computer systems, minicomputers, mainframe computers, as well as personal computers, hand-held computing devices, microprocessor-based or programmable consumer electronics, and the like, each of which can be operatively coupled to one or more associated devices.

The illustrated aspects of the claimed subject matter may also be practiced in distributed computing environments where certain tasks are performed by remote processing devices that are linked through a communications network. In a distributed computing environment, program modules can be located in both local and remote memory storage devices.

A computer typically includes a variety of computer-readable media. Computer-readable media can be any available media that can be accessed by the computer and includes both volatile and non-volatile media, removable and non-removable media. By way of example, and not limitation, computer-readable media can comprise computer storage media and communication media. Computer storage media includes both volatile and non-volatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital video disk (DVD) or other optical disk storage, magnetic cassettes, magnetic tape, magnetic disk storage or other magnetic storage devices, or any other medium which can be used to store the desired information and which can be accessed by the computer.

With reference again to FIG. 11, the illustrative environment 1100 for implementing various aspects includes a computer 1102, the computer 1102 including a processing unit 1104, a system memory 1106 and a system bus 1108. The system bus 1108 couples system components including, but not limited to, the system memory 1106 to the processing unit 1104. The processing unit 1104 can be any of various commercially available processors. Dual microprocessors and other multi-processor architectures may also be employed as the processing unit 1104.

The system bus 1108 can be any of several types of bus structure that may further interconnect to a memory bus (with or without a memory controller), a peripheral bus, and a local bus using any of a variety of commercially available bus architectures. The system memory 1106 includes read-only memory (ROM) 1110 and random access memory (RAM) 1112. A basic input/output system (BIOS) is stored in a non-volatile memory 1110 such as ROM, EPROM, EEPROM, which BIOS contains the basic routines that help to transfer information between elements within the computer 1102, such as during start-up. The RAM 1112 can also include a high-speed RAM such as static RAM for caching data.

The computer 1102 further includes an internal hard disk drive (HDD) 1114 (e.g., EIDE, SATA), which internal hard disk drive 1114 may also be configured for external use in a suitable chassis (not shown), a magnetic floppy disk drive (FDD) 1116, (e.g., to read from or write to a removable diskette 1118) and an optical disk drive 1120, (e.g., reading a CD-ROM disk 1122 or, to read from or write to other high capacity optical media such as the DVD). The hard disk drive 1114, magnetic disk drive 1116 and optical disk drive 1120 can be connected to the system bus 1108 by a hard disk drive interface 1124, a magnetic disk drive interface 1126 and an optical drive interface 1128, respectively. The interface 1124 for external drive implementations includes at least one or both of Universal Serial Bus (USB) and IEEE 1094 interface technologies. Other external drive connection technologies are within contemplation of the claimed subject matter.

The drives and their associated computer-readable media provide nonvolatile storage of data, data structures, computer-executable instructions, and so forth. For the computer 1102, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the illustrative operating environment, and further, that any such media may contain computer-executable instructions for performing the methods of the disclosed and claimed subject matter.

A number of program modules can be stored in the drives and RAM 1112, including an operating system 1130, one or more application programs 1132, other program modules 1134 and program data 1136. All or portions of the operating system, applications, modules, and/or data can also be cached in the RAM 1112. It is to be appreciated that the claimed subject matter can be implemented with various commercially available operating systems or combinations of operating systems.

A user can enter commands and information into the computer 1102 through one or more wired/wireless input devices, e.g., a keyboard 1138 and a pointing device, such as a mouse 1140. Other input devices (not shown) may include a microphone, an IR remote control, a joystick, a game pad, a stylus pen, touch screen, or the like. These and other input devices are often connected to the processing unit 1104 through an input device interface 1142 that is coupled to the system bus 1108, but can be connected by other interfaces, such as a parallel port, an IEEE 1094 serial port, a game port, a USB port, an IR interface, etc.

A monitor 1144 or other type of display device is also connected to the system bus 1108 via an interface, such as a video adapter 1146. In addition to the monitor 1144, a computer typically includes other peripheral output devices (not shown), such as speakers, printers, etc.

The computer 1102 may operate in a networked environment using logical connections via wired and/or wireless communications to one or more remote computers, such as a remote computer(s) 1148. The remote computer(s) 1148 can be a workstation, a server computer, a router, a personal computer, portable computer, microprocessor-based entertainment appliance, a peer device or other common network node, and typically includes many or all of the elements described relative to the computer 1102, although, for purposes of brevity, only a memory/storage device 1150 is illustrated. The logical connections depicted include wired/wireless connectivity to a local area network (LAN) 1152 and/or larger networks, e.g., a wide area network (WAN) 1154. Such LAN and WAN networking environments are commonplace in offices and companies, and facilitate enterprise-wide computer networks, such as intranets, all of which may connect to a global communications network, e.g., the Internet.

When used in a LAN networking environment, the computer 1102 is connected to the local network 1152 through a wired and/or wireless communication network interface or adapter 1156. The adaptor 1156 may facilitate wired or wireless communication to the LAN 1152, which may also include a wireless access point disposed thereon for communicating with the wireless adaptor 1156.

When used in a WAN networking environment, the computer 1102 can include a modem 1158, or is connected to a communications server on the WAN 1154, or has other means for establishing communications over the WAN 1154, such as by way of the Internet. The modem 1158, which can be internal or external and a wired or wireless device, is connected to the system bus 1108 via the serial port interface 1142. In a networked environment, program modules depicted relative to the computer 1102, or portions thereof, can be stored in the remote memory/storage device 1150. It will be appreciated that the network connections shown are illustrative and other means of establishing a communications link between the computers can be used.

The computer 1102 is operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi and Bluetooth™ wireless technologies. Thus, the communication can be a predefined structure as with a conventional network or simply an ad hoc communication between at least two devices.

Wi-Fi, or Wireless Fidelity, allows connection to the Internet from a couch at home, a bed in a hotel room, or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11x (a, b, g, etc.) to provide secure, reliable, fast wireless connectivity. A Wi-Fi network can be used to connect computers to each other, to the Internet, and to wired networks (which use IEEE 802.3 or Ethernet).

Wi-Fi networks can operate in the unlicensed 2.4 and 5 GHz radio bands. IEEE 802.11 applies to generally to wireless LANs and provides 1 or 2 Mbps transmission in the 2.4 GHz band using either frequency hopping spread spectrum (FHSS) or direct sequence spread spectrum (DSSS). IEEE 802.11a is an extension to IEEE 802.11 that applies to wireless LANs and provides up to 54 Mbps in the 5 GHz band. IEEE 802.11a uses an orthogonal frequency division multiplexing (OFDM) encoding scheme rather than FHSS or DSSS. IEEE 802.11b (also referred to as 802.11 High Rate DSSS or Wi-Fi) is an extension to 802.11 that applies to wireless LANs and provides 11 Mbps transmission (with a fallback to 5.5, 2 and 1 Mbps) in the 2.4 GHz band. IEEE 802.11g applies to wireless LANs and provides 20+ Mbps in the 2.4 GHz band. Products can contain more than one band (e.g., dual band), so the networks can provide real-world performance similar to the basic 10BaseT wired Ethernet networks used in many offices.

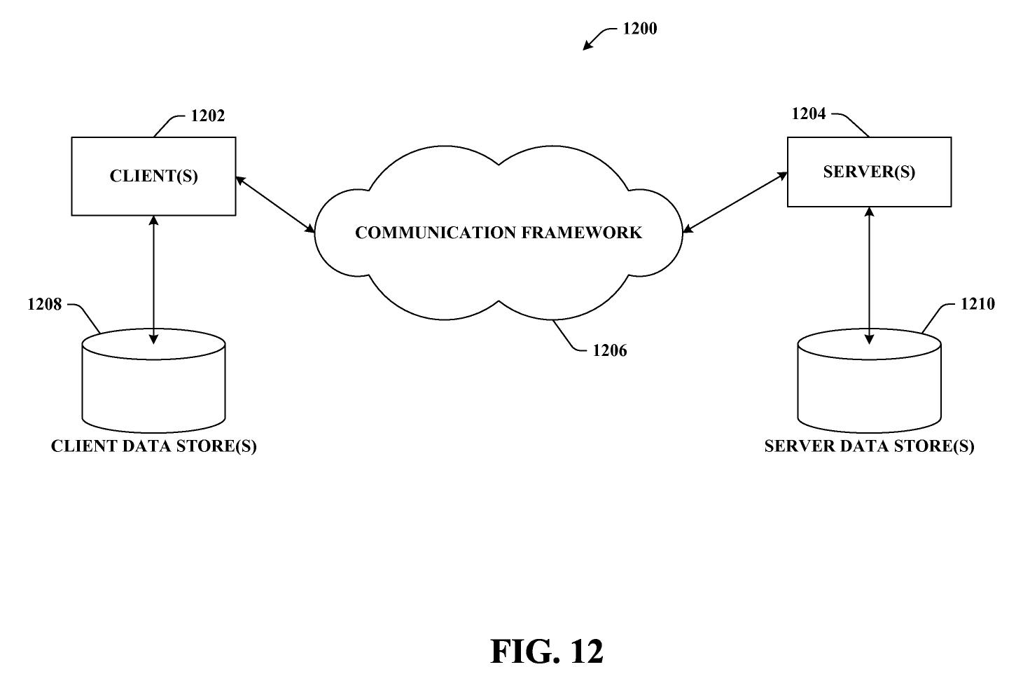

Referring now to FIG. 12, there is illustrated a schematic block diagram of an illustrative computing environment 1200 for processing the disclosed architecture in accordance with another aspect. The system 1200 includes one or more client(s) 1202. The client(s) 1202 can be hardware and/or software (e.g., threads, processes, computing devices). The client(s) 1202 can house cookie(s) and/or associated contextual information by employing the claimed subject matter, for example.

The system 1200 also includes one or more server(s) 1204. The server(s) 1204 can also be hardware and/or software (e.g., threads, processes, computing devices). The servers 1204 can house threads to perform transformations by employing the claimed subject matter, for example. One possible communication between a client 1202 and a server 1204 can be in the form of a data packet adapted to be transmitted between two or more computer processes. The data packet may include a cookie and/or associated contextual information, for example. The system 1200 includes a communication framework 1206 (e.g., a global communication network such as the Internet) that can be employed to facilitate communications between the client(s) 1202 and the server(s) 1204.

Communications can be facilitated via a wired (including optical fiber) and/or wireless technology. The client(s) 1202 are operatively connected to one or more client data store(s) 1208 that can be employed to store information local to the client(s) 1202 (e.g., cookie(s) and/or associated contextual information). Similarly, the server(s) 1204 are operatively connected to one or more server data store(s) 1210 that can be employed to store information local to the servers 1204.

What has been described above includes examples of the disclosed and claimed subject matter. It is, of course, not possible to describe every conceivable combination of components and/or methodologies, but one of ordinary skill in the art may recognize that many further combinations and permutations are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A machine implemented system that provides biometric feedback monitoring of brain activity, comprising:
a component that received via an interface an indication of brain activity associated with a brain of an individual, the component employs the indication of brain activity to ensure that the individual is maximizing brain activity, where the component determines the indication of brain activity is suboptimal the component sends feedback to the individual to stimulate brain activity.

2. The system of claim 1, the feedback includes assigning the individual a task that stimulates a specific response center of the brain.

3. The system of claim 1, the component monitors brain activity to determine an amount of time that the individual dwells on contemplating a particular topic.

4. The system of claim 1, the component tracks brain activity to ascertain relationships between a reaction of the individual to a supplied stimulus perceived by the individual.

5. The system of claim 1, the component utilizes an aversion associated with a stimulus indicated by the individual to activate a response center of the brain.

6. The system of claim 1, the component accesses a health record associated with the individual and persisted on a health manager by communicating a cryptographic key supplied by the individual.

7. The system of claim 1, the component effectuates a vertical search of a health manager based at least in part on a health record associated with the individual.

8. A machine implemented method that provides biometric feedback monitoring of brain activity, comprising:
obtaining an indication of brain activity associated with a brain of an individual;
based at least in part on the indication of brain activity determining whether the individual is maximizing brain activity;
based at least in part on the determining ascertaining whether the indication of brain activity is suboptimal; and
directing feedback to the individual where the ascertaining indicates suboptimal brain activity.

9. The method of claim 8, further comprising monitoring brain activity to determine an amount of time that the individual dwells on a particular topic.

10. The method of claim 8, further comprising tracking brain activity to ascertain a relationship between an aversion to a supplied stimulus as perceived by the individual.

11. The method of claim 10, further comprising utilizing the aversion to activate a response center of the brain.

12. The method of claim 8, further comprising accessing a health record associated with the individual and persisted on a health manager by communicating at least one of cryptographic key, a username and password couplet, or a biometric attribute inherently associated with or supplied by the individual.

13. The method of claim 12, further comprising utilizing the health record associated with the individual to effectuate a vertical search of the health manager.

14. A system that provides biometric feedback monitoring of brain activity, comprising:
means for determining maximal brain activity based at least in part on an indication of brain activity associated with a brain of an individual; and
means for directing feedback to the individual where the means for determining indicates suboptimal brain activity.

15. The system of claim 14, the feedback includes assigning the individual a task that stimulates a specific response center of the brain.

16. The system of claim 14, further comprising means for monitoring brain activity to determine an amount of time that the individual dwells on a topic, the amount of time utilized to provide feedback to the individual.

17. The system of claim 14, the means for directing feedback utilized where the means for determining indicates brain activity is more than optimal,

18. The system of claim 17, the feedback includes assigning the individual an activity that calms a specific response center of the brain.

19. The system of claim 14, further comprising means for recording brain activity to ascertain a relationship between a reaction of the individual and an unpleasant stimulus.

20. The system of claim 20, the relationship between the reaction of the individual and the unpleasant stimulus utilized to at least one of calm or stimulate the brain.
